(19) United States
(12) Patent Application Publication
(10) Pub. No.: US 2023/0091866 A1
(43) Pub. Date: Mar. 23, 2023

(54) Method And System For Non-Invasive Detection Of A Living Subject's Blood Oxygen Saturation

(71) Applicant: Miku, Inc., Woodbridge, NJ (US)

(72) Inventors: Eric Gregory White, Tinton Falls, NJ (US); David Robert Abrams, Aberdeen, NJ (US)

(73) Assignee: Miku, Inc., Woodbridge, NJ (US)

(21) Appl. No.: 17/989,804

(22) Filed: Nov. 18, 2022

Related U.S. Application Data
(63) Continuation-in-part of application No. 17/902,566, filed on Sep. 2, 2022.
Application No. 17/902,566 is a continuation-in-part of application No. 17/887,426, filed on Aug. 13, 2022.
Application No. 17/887,426 is a continuation-in-part of application No. 17/872,952, filed on Jul. 25, 2022.
(60) Provisional application No. 63/281,695, filed on Nov. 21, 2021.
(60) Provisional application No. 63/241,539, filed on Sep. 8, 2021.
(60) Provisional application No. 63/233,388, filed on Aug. 16, 2021.
(60) Provisional application No. 63/226,703, filed on Jul. 28, 2021.

(51) Int. Cl. A61B 5/00

(52) U.S. Cl. CPC A61B 5/0064; A61B 5/0082

(57) ABSTRACT

A method and system for non-invasive detection of a living subject's blood oxygen saturation is disclosed herein. A system, method, and apparatus utilizes an imaging RGB/infrared sensor and an active two-color light source for detecting blood oxygen saturation (SpO2) of a living subject in a non-contact manner. The system comprises a non-contact light source comprising red light or infrared light; an imaging sensor; a processor; and a user interface.

### CROSS REFERENCES TO RELATED APPLICATIONS

The Present Application claims priority to U.S. Provisional Patent Application No. 63/281,695, filed on Nov. 21, 2021, and is a continuation-in-part application of U.S. patent application Ser. No. 17/902,566, filed on Sep. 2, 2022, which claims priority to U.S. Provisional Patent Application No. 63/241,539, filed on Sep. 8, 2021, now expired, and is a continuation-in-part application of U.S. patent application Ser. No. 17/887,426, filed on Aug. 13, 2022, which claims priority to U.S. Provisional Patent Application No. 63/233,388 filed on Aug. 16, 2021, now expired, and is a continuation-in-part application of U.S. patent application Ser. No. 17/872,952, filed on Jul. 25, 2022, which claims priority to U.S. Provisional Patent Application No. 63/226,703, filed on Jul. 28, 2021, now expired, each of which is hereby incorporated by reference in its entirety.

### STATEMENT REGARDING FEDERALLY SPONSORED RESEARCH OR DEVELOPMENT

Not Applicable

### BACKGROUND OF THE INVENTION

### Field of the Invention

The present invention generally relates to detecting blood oxygen saturation (SpO2) of a living target.

### Description of the Related Art

The typical method for measuring blood oxygen saturation (SpO2) in a living subject is a two-color red/infrared contact sensor which makes contact with a translucent part of the subject's skin such as a finger, wrist, foot, or ear lobe. The current market has provided only products which require contact with the target in order to provide these vitals measurements. This poses a risk for infection in immunosuppressed targets, and there is a risk of losing signal continuity in instances where the target removes the sensor.

Existing SpO2 monitoring systems rely on a technology solution which requires physical contact with the subject's skin and relies on the subject's ignorance of, or compliance with, the system. For subjects such as babies, athletes, or hospital inpatients, the only available sensor is one which generates red and infrared lighting in close contact with a translucent part of the skin, such as a finger, wrist, foot, or ear lobe. The amount of red light reflected or absorbed compared to the amount of IR light reflected or absorbed is then used to estimate the SpO2 level of the target.

In order to provide continuous monitoring of the patient, these existing sensors must maintain physical contact the entire time. This means if the sensor loses contact with the skin for any reason, the subject is no longer being monitored. These sensors can lose contact with the patient's skin in many instances, such as if the baby kicks off an SpO2 sock, or a fussy sleeper takes off their SpO2 tracking watch, or a hospital inpatient's SpO2 finger sensor slips off of their finger. In instances where it is desired to continuously monitor a patient's SpO2, a new technology solution is required.

### BRIEF SUMMARY OF THE INVENTION

The present invention provides a system in which monitoring of a patient's blood oxygen saturation (SpO2) level does not require direct continuous contact with the skin and allows for uninterrupted monitoring.

The present invention is a system, method, and apparatus for using an imaging RGB (or just R) and an active two-color light source for detecting blood oxygen saturation (SpO2) of a living target.

One aspect of the present invention is a method for non-invasive detection of a living subject's blood oxygen saturation. The method includes focusing light from a non-contact light source on the living subject, the light selected from red light or infrared light. The method also includes detecting reflected light from the living subject at an imaging sensor. The method also includes transmitting reflected light data from the imaging sensor to a processor for processing to determine the living subject's blood oxygen saturation value. The method also includes transmitting the living subject's blood oxygen saturation value from the processor to a user interface for communication.

Another aspect of the present invention is a system for non-invasive detection of a living subject's blood oxygen saturation. The system comprises a non-contact light source comprising red light or infrared light; an imaging sensor; a processor; and a user interface. The non-contact light source is configured to focus light on the living subject. The imaging sensor is configured to detect reflected light from the living subject. The imaging sensor is configured to transmit the reflected light data to a processor. The processor is configured to determine the living subject's blood oxygen saturation value. The processor is configured to transmit the living subject's blood oxygen saturation value to the user interface for communication.

Yet another aspect of the present invention is a method for non-invasive detection of a living subject's blood oxygen saturation in a room. The method includes detecting a presence of a living subject in a room utilizing an object detection algorithm running on a processor of a sensor system within the room. The method also includes identifying an area of the living subject's skin using an image analysis search algorithm running on the processor of the sensor system. The method also includes focusing light from a non-contact light source on the living subject, the light selected from red light or infrared light. The method also includes detecting reflected light from the living subject at an imaging sensor. The method also includes transmitting reflected light data from the imaging sensor to a processor for processing to determine the living subject's blood oxygen saturation value. The method also includes transmitting the living subject's blood oxygen saturation value from the processor to a user interface for communication.

Having briefly described the present invention, the above and further objects, features and advantages thereof will be recognized by those skilled in the pertinent art from the following detailed description of the invention when taken in conjunction with the accompanying drawings.

### BRIEF DESCRIPTION OF THE SEVERAL VIEWS OF THE DRAWINGS

FIG. 1 is an illustration of blood oxygen saturation measurement.

FIG. 2 is a block diagram of a system of the present invention.

FIG. 3 is a block diagram of a system of the present invention.

FIG. 4 is a flow chart of a general method of the present invention.

### DETAILED DESCRIPTION OF THE INVENTION

A system, method, and apparatus which consists of an optical sensor such as a RGB/IR or red- and infrared-sensitive camera or detector, along with an active two-color lighting system, and a set of algorithms which operate internally on the system, which determine and transmit presence and blood oxygen saturation (SpO2) information to an observer. Optical sensor can be imaging, or can be a single-pixel detector with a targeting method such as a gimbal.

The best way to mitigate these problems is by detecting and measuring these vital signs remotely and without making contact with the target. This is preferably performed by controlling the surrounding lighting environment with calibrated light sources of known frequencies and capturing the reflections in a calibrated imaging camera, and using an algorithm to compute the SpO2 using known optical scattering properties of blood oxygen saturation.

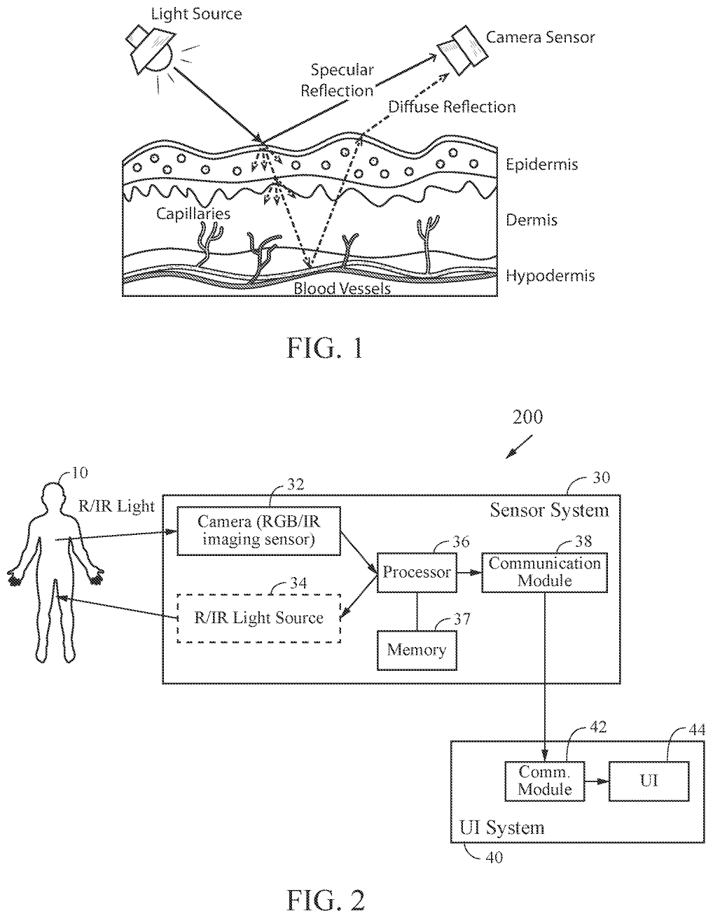

“Pulse oximetry is based on the technique of photoplethysmography, and takes advantage of the fact that oxyhaemoglobin (HbO2) and reduced haemoglobin (Hb) absorb light differently at different wavelengths; that arterial blood is mostly pulsatile in nature; and that an optical window exists in the far visible and short-wave infrared for water which allows radiation in this wavelength range to probe the vasculature of the dermis” (Guazzi et al, at the www website ncbi.nlm.nih.gov/pmc/articles/PMC4574660/), as shown in FIG. 1.

Two-color light source can be generated using any manner of lighting generation solutions, including an ambient lighting source such as ambient lighting ring or nightlight-style lighting source, a more directed light source such as a mirror directed laser or laser on gimbal, or a more directed “spotlight” source with restricted beam divergence.

When SpO2 drops below a specified threshold value such as 90%, a notification or alarm can be sent to an observer.

A system for non-invasive detection of a living subject's blood oxygen saturation 200, shown in FIG. 2, preferably comprises a sensor system 30 and a user interface system 40. The sensor system 30 comprises a RGB/IR imaging sensor 32, a red/infrared light source 34, a processor 36, a memory 37 and a communication module 38. The user interface system 40 preferably comprises a communication module 42 and a user interface 44.

An RGB/SW IR imaging sensor 32 is preferably used to detect light reflected by a living subject from ambient (e.g., sunlight, room lighting, etc.) and controlled (e.g., light bulb, SW IR LED, etc.) light sources 34.

The field of view of the imaging sensor and the range of motion or attitude of the light source are roughly collocated with similar attitude (azimuth and zenith angle) or their offsets calibrated.

Digital sensor data from these sensor modules are provided for ingestion by a processor unit 36, which runs an algorithm internally to perform digital signal processing, feature extraction, decision logic, and preparation for communication with the user's module. Data is then transmitted from the sensor system communication module 38 to the corresponding user interface system's 40 communication module 42.

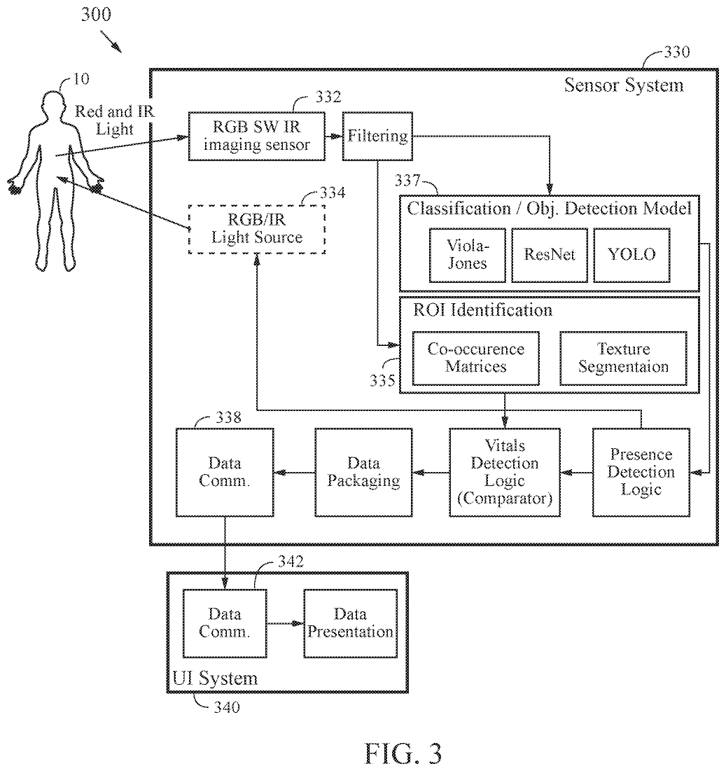

With reference to FIG. 3, an object detection algorithm 337 of a sensor system 330 trained on a class of subjects is used in order to detect the presence of a subject 10. An image analysis search algorithm 335 such as texture segmentation or ROI calculation is used to identify areas of a patient's skin. When the subject 10 is detected, an algorithm controls the red and infrared light sources 334 such that the identified areas of the patient's skin are illuminated by the red and infrared light sources. This is preferably done by controlling the azimuth and elevation angle of the light source via a rotation control such as a gimbal, or by controlling the reflected angle of rotation using a mirror with rotation control, or by flooding the subject with light. One such algorithm would alternate the presence of red and infrared light such that each is enabled for one second before the other is enabled. The imaging sensor 332 then captures an image when each light source is at full strength and compares the reflected light of such areas. The data is filtered and processed. The ratio of reflected light is then used to estimate the patient's blood oxygen saturation (SpO2) using an algorithm such as Sophia (Skin-Oxygen Photoplethysmographic Image Analysis). The data is sent to a vitals detection logic (a comparator), then packaged and sent to a data communication module 338 for transmission (wired or wirelessly) to a data communication module 342 of a user interface system 342.

Although an absolute measure of oxygen saturation is not claimed (this will depend on factors such as the lighting and the skin color of the subject), the method is shown to be able to track changes in oxygen saturation changes with accuracy comparable to that of a conventional pulse oximeter. This relative oxygen saturation is then sent to a user interface 340.

When relative oxygen saturation drops below 90% a notification or alarm may also be sent to the user interface.

The user interface system 340 preferably comprises a data communication module 342 and a user interface.

The data communication module 342 receives data from its corresponding sensor system. This data is presented to the user via a user interface such as an LED, a display, a speaker, or any other manner of interface.

A preferred example of skin detection is as follows: collect image I[t] output with sample rate S Hz or when otherwise requested by system, S could be 0.2 Hz for example; perform face detection model inference using trained model, example trained model includes R-CNN model or Viola Jones; and use response from model to update status of baby skin location.

A preferred example of SpO2 computation (imaging sensor) is as follows: configure R/IR light source to transmit red light; using imaging R/IR detector, capture image I1; configure R/IR light source to transmit IR light; using imaging R/IR detector, capture image I2; calculate image difference D; isolate region of image identified in skin detection method; and compute ratio of the average of each pixel's value.

A preferred example of SpO2 alert computation is as follows: using method described above in SpO2 Computation, SpO2 level s[n] is computed for a target every k seconds for N minutes (for example, k=60 and N=60); the median value s_median is computed over s[n] and used as the baseline measurement; s[n] is continued to be observed every k seconds; and if s[n] drops below a certain threshold M*s_median for some M such as M=0.90, send an alert to the user.

As shown in FIGS. 2 and 3, a system 200/300 for non-invasive detection of a living subject's 10 blood oxygen saturation comprises a non-contact light source comprising red light or infrared light 34/334, an imaging sensor 30/330, a processor 36, and a user interface 40/340.

A sensor system 30/330 consists of an RGB/SW IR imaging sensor 32/332, used to detect light reflected by a living subject 10 from ambient (e.g., sunlight, room lighting, etc.) and controlled (e.g., light bulb, SW IR LED, etc.) light sources.

The RGB/IR imaging sensor 32/332 determines presence of a living subject 10 and vitals under certain circumstances. The RGB/IR imaging sensor 32/332 also determines the magnitude of reflected red and infrared light.

The Processing module 36, with memory 37 and communication module 38, performs the entirety of presence and vitals detection within the sensing system.

In reference to FIG. 3, a user interface system 340 with communication module 342 receives data and presents information to a user.

In reference to FIG. 2, a user interface system 40 consists of a communication module 42 and a user interface 44. The communication module 42 receives data from its corresponding module 38 in the sensor system 30. This data is presented to the user via a user interface 44 such as an LED, a display, a speaker, or any other manner of interface.

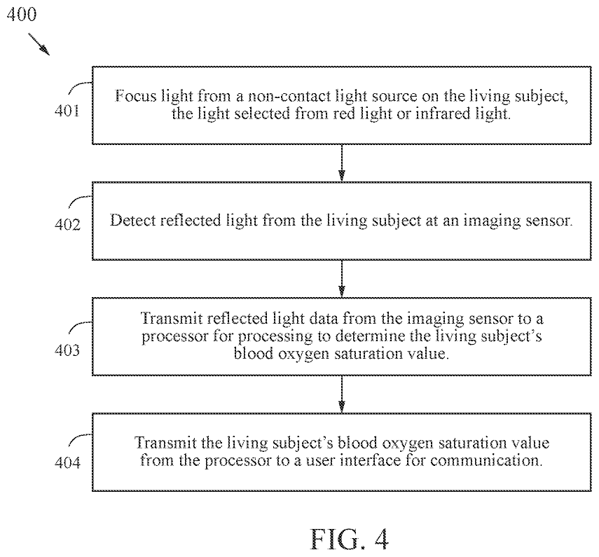

In one embodiment, a system for non-invasive detection of a living subject's SpO2 saturation comprises a non-contact light source comprising red light or infrared light, an imaging sensor, a processor, and a user interface. In reference to FIG. 4, a flow chart for a method 400 for non-invasive detection of a living subject's blood oxygen saturation, step 401 is the non-contact light source is configured to focus light on the living subject. The light is preferably selected from red or infrared light. The imaging sensor is configured to detect reflected light from the living subject at step 402. The imaging sensor is also configured to transmit the reflected light data to a processor at step 403. The processor is configured to determine the living subject's blood oxygen saturation value. In step 404 the processor is further configured to transmit the living subject's blood oxygen saturation value to the user interface for communication.

White et al., U.S. Pat. No. 10,825,314 for a Baby Monitor, is hereby incorporated by reference in its entirety.

From the foregoing it is believed that those skilled in the pertinent art will recognize the meritorious advancement of this invention and will readily understand that while the present invention has been described in association with a preferred embodiment thereof, and other embodiments illustrated in the accompanying drawings, numerous changes modification and substitutions of equivalents may be made therein without departing from the spirit and scope of this invention which is intended to be unlimited by the foregoing except as may appear in the following appended claim. Therefore, the embodiments of the invention in which an exclusive property or privilege is claimed are defined in the following appended claims.

We claim as our invention the following:

1. A method for non-invasive detection of a living subject's blood oxygen saturation, the method comprising:
detecting a presence of a living subject in a room utilizing an object detection algorithm running on a processor of a sensor system within the room;
identifying an area of the living subject's skin using an image analysis search algorithm running on the processor of the sensor system;
focusing light from a non-contact light source on the living subject, the light selected from red light or infrared light;
detecting reflected light from the living subject at an imaging sensor;
transmitting reflected light data from the imaging sensor to a processor for processing to determine the living subject's blood oxygen saturation value; and
transmitting the living subject's blood oxygen saturation value from the processor to a user interface for communication.

2. The method according to claim 1 wherein the processor which runs an algorithm internally to perform digital signal processing, feature extraction, decision logic, and preparation for communication with the user's module.

3. The method according to claim 1 further comprising alternating the use of red light or infrared light.

4. The method according to claim 1 wherein the image analysis search algorithm is selected from the group of a texture segmentation or a ROI calculation.

5. The method according to claim 4 further comprising controlling the azimuth and elevation angle of the light source based on identifying the area of the living subject's skin.

6. The method according to claim 4 further comprising controlling a reflected angle of rotation using a mirror with rotation control.

7. The method according to claim 4 further comprising flooding the subject with light.

8. The method according to claim 8 further comprising capturing an image when each light source is at full strength and comparing the reflected light of the area of the living subject's skin.

9. The method according to claim 9 further comprising using a ratio of reflected light to estimate the living subject's blood oxygen saturation (SpO2) using a Sophia (Skin-Oxygen Photoplethysmographic Image Analysis) algorithm.

10. A system for non-invasive detection of a living subject's blood oxygen saturation, the system comprising:
a non-contact light source comprising red light or infrared light;
an imaging sensor;
a processor; and
a user interface;
wherein the processor is configured to run object detection algorithm trained to detect the presence of a living subject in a room;
wherein non-contact light source is configured to focus light on the living subject;
wherein the imaging sensor is configured to detect reflected light from the living subject;
wherein the imaging sensor is configured to transmit the reflected light data to a processor;
wherein the processor is configured to determine the living subject's blood oxygen saturation value; and
wherein the processor is configured to transmit the living subject's blood oxygen saturation value to the user interface for communication.

11. The system according to claim 10 wherein the processor is configured to run an algorithm internally to perform digital signal processing, feature extraction, decision logic, and preparation for communication with the user's module.

12. The system according to claim 10 wherein the light source is configured to alternate the use of red light or infrared light.

13. The system according to claim 10 wherein imaging sensor is configured to identify an area of the living subject's skin using an image analysis search algorithm selected from the group of a texture segmentation or a ROI calculation.

14. The system according to claim 14 wherein the light source is configured to control the azimuth and elevation angle of the light source based on identifying the area of the living subject's skin.

15. The system according to claim 14 further comprising a mirror with rotation control configured to control a reflected angle of rotation using.

16. The system according to claim 14 wherein the light source is configured to flood the subject with light.

17. The system according to claim 16 wherein the imaging sensor is configured to capture an image when each light source is at full strength and comparing the reflected light of the area of the living subject's skin.

18. The system according to claim 17 wherein the processor is configured to use a ratio of reflected light to estimate the living subject's blood oxygen saturation (SpO2) using a Sophia (Skin-Oxygen Photoplethysmographic Image Analysis) algorithm.
